(19) United States
(12) Patent Application Publication
(10) Pub. No.: US 2025/0058053 A1
(43) Pub. Date: Feb. 20, 2025

(54) OXYGEN SCAVENGER SYRINGE CAP

(71) Applicant: Fresenius Kabi USA, LLC, Lake Zurich, IL (US)

(72) Inventors: Christopher J. MURRAY, Lake Zurich, IL (US); Anthony D. FERRARO, Lake Zurich, IL (US)

(21) Appl. No.: 18/720,828

(22) PCT Filed: Dec. 13, 2022
(86) PCT No.: PCT/US2022/081453

§ 371 Date: Jun. 17, 2024

Related U.S. Application Data
(60) Provisional application No. 63/292,039, filed on Dec. 21, 2021.

(51) Int. Cl. A61M 5/315; A61M 5/31

(52) U.S. Cl. CPC A61M 5/31513; A61M 2005/3104; A61M 2005/3118; A61M 2005/312

(57) ABSTRACT

The present disclosure describes a syringe assembly including a syringe and an oxygen scavenger syringe cap disposed over a connection of the syringe, the oxygen scavenger syringe cap having a main body that defines a proximal end defining an opening, a distal end opposite the proximal end along the axial direction, an outer surface, and an inner surface opposite the outer surface that defines a passage to receive a tip cap and the connection, where the oxygen scavenger syringe cap is spaced from the tip cap. The oxygen scavenger syringe cap further includes an oxygen absorber.

### CROSS REFERENCE TO RELATED APPLICATIONS

This application claims the benefit of U.S. Provisional Patent App. No. 63/292,039, filed Dec. 21, 2021, the disclosure of which is hereby incorporated by reference herein.

### TECHNICAL FIELD

The present disclosure generally relates to oxygen scavenger devices, and, more particularly, to an oxygen scavenger syringe cap for syringes filled with a material.

### BACKGROUND

Syringe assemblies are used to hold, transport, and deliver materials. For example, syringes are often utilized in medical environments to administer one or more medicinal materials. Syringe assemblies may differ in size, and their specific dimensions are dictated by the desired application and the specific material to be administered. In some instances, syringes may be pre-filled with one or more materials that are then dispensed from the syringe and combined with other elements.

Medicinal materials include oxygen sensitive drugs. The shelf life of oxygen sensitive drugs is extended when oxygen is removed from the syringe assembly. This is especially the case where the syringe assembly includes an oxygen permeable tip cap. Existing technology for reducing oxygen from a medical material is directed at packaging systems, external of the syringe assembly. These systems do not allow for the syringe assembly to reduce oxygen levels once the packaging is removed, and may be cumbersome within the medical environment. Further, the addition of an oxygen reducing element may often require changes to be made to the manufacturing process of a syringe or its constituent components, which increases associated production costs and complexity of manufacturing.

Therefore, there is a need for an oxygen scavenger cap configured to be used with syringes having preexisting designs that are filled with a material.

### SUMMARY

An embodiment of the present disclosure is a syringe assembly comprising a syringe having a barrel body that extends from a proximal end to a distal end and defines a chamber extending along an axial direction therethrough, and a connection extending from the distal end along the axial direction and defining an outlet in fluid communication with the chamber, where the chamber contains a material. The syringe assembly includes a plunger received within the chamber of the syringe to create a fluid seal within the barrel body and a tip cap defining a central passage configured to receive a portion of the connection such that the tip cap creates a fluid seal over the outlet. The syringe assembly also includes an oxygen scavenger syringe cap disposed over the connection, the oxygen scavenger syringe cap having a main body that defines a proximal end defining an opening, a closed distal end opposite the proximal end along the axial direction, an outer surface, and an inner surface opposite the outer surface that defines a passage configured to receive the tip cap and the connection, where the oxygen scavenger syringe cap comprises an oxygen absorber.

Another embodiment of the present disclosure is a method of filling a syringe with a material, where the method comprises receiving a syringe having a barrel body extending from a distal end having a connection defining an outlet to an open proximal end, the barrel body defining a chamber that extends along an axial direction therethrough, where a tip cap is placed over the outlet to create a fluid seal over the outlet. The method includes filling the chamber with the material through the open proximal end, disposing a plunger within the chamber at the proximal end, and applying an oxygen scavenger syringe cap over the connection and the tip cap.

Another embodiment of the present disclosure is an oxygen scavenger syringe cap that includes a main body having a proximal end defining an opening, a closed distal end opposite the proximal end along an axial direction, an outer surface, and an inner surface opposite the outer surface. The inner surface defines a passage configured to receive a tip cap such that the tip cap is entirely spaced from the oxygen scavenger syringe cap. The inner surface is configured to contact a connection of the syringe. The oxygen scavenger syringe cap also comprises an oxygen absorber.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates a perspective view of a syringe assembly in accordance with an embodiment of the present disclosure;

FIG. 2 illustrates a perspective view of a plunger rod and plunger of the syringe assembly shown in FIG. 1;

FIG. 3 illustrates a perspective view of a syringe of the syringe assembly shown in FIG. 1;

FIG. 4A illustrates a side view of the syringe assembly shown in FIG. 1 with certain components removed for clarity, with the plunger and plunger rod in a first position;

FIG. 4B illustrates a side view of the syringe assembly shown in FIG. 1 with certain components removed for clarity, with the plunger and plunger rod in a second position;

FIG. 5 illustrates an exploded view of a distal portion of the syringe assembly shown in FIG. 1;

FIG. 6 illustrates a side view of an oxygen scavenger syringe cap of the syringe assembly shown in FIG. 1;

FIG. 7 illustrates a cross-sectional view of a distal portion of the syringe assembly shown in FIG. 1, taken along line 7-7 shown in FIG. 1;

FIG. 8 illustrates a process flow diagram of a method of filling a syringe with a material according to an embodiment of the present disclosure;

### DETAILED DESCRIPTION OF ILLUSTRATIVE EMBODIMENTS

A feature of the syringe assemblies herein is that the configuration allows for the absorption and removal of oxygen in all components of a syringe assembly, and especially from the tip cap. The oxygen absorber removes the oxygen from the syringe assembly through the tip cap, which comprises an oxygen permeable material such as an elastomer. Suitable elastomers include, for example, vulcanized elastomers and styrenic block copolymer thermoplastic elastomers, but also natural rubber, acrylate-butadiene rubber, cis-polybutadiene, chlroro or bromobutyl rubber, chlorinated polyethylene elastomers, polyalkylene oxide polymers, ethylene vinyl acetate, fluorosilicone rubbers, hexafluoropropylene-vinylidene fluoride-tetrafluoroethylene terpolymers, butyl rubbers, polyisobutene, synthetic polyisoprene rubber, silicone rubbers, styrene-butadiene rubbers, tetrafluoroethylene propylene copolymers, thermoplastic-copolyesters, thermo-plastic elastomers, or a combination thereof. The oxygen permeable material allows for sterilization processes via gas (e.g., ethylene oxide) or steam sterilization of the syringe and the material within the syringe assembly. The tip cap can be a single material tip cap or a bi-material tip cap, e.g., a plastic and an elastomer. The oxygen absorber may also remove the oxygen from the material within the syringe assembly. Over time, the residual oxygen amount that is present within the syringe assembly is reduced and even eliminated. As a result, the syringe assembly systems described herein offer an increase in the shelf life of oxygen sensitive drugs past conventional syringe assembly systems. In some embodiments, the syringe assembly described herein may maintain about 0% oxygen level in the syringe for at least 12 months, although the about 0% oxygen level may be maintained through up to 48 months.

Syringes generally comprise a cylindrical barrel, often made of glass but more recently have been made of plastic materials, for example, cyclic olefin polymers or acrylonitrile butadiene styrene (ABS), polycarbonate (PC), polyoxymethylene (POM), polystyrene (PS), polybutylene terephthalate (PBT), polypropylene (PP), polyethylene (PE), polyamide (PA), thermoplastic elastomer (TPE) and their combinations. The barrels of such syringes are operated with an elastomer plunger which can be urged along the barrel to eject liquid content via a nozzle. Suitable elastomers for such plungers may be based on the same elastomers as mentioned above for tip caps.

Described herein is a syringe assembly 10 that includes an oxygen scavenger syringe cap 200. Certain terminology is used to describe the syringe assembly 10 in the following description for convenience only and is not limiting. The words “right,” “left,” “lower,” “upper,” “proximal,” and “distal” designate directions in the drawings to which reference is made. The words “inner” and “outer” refer to directions toward and away from, respectively, the geometric center of the description to describe the syringe assembly 10 and related parts thereof. The words “axially” and “radially” refer to directions along the orthogonal axial and radial directions A, R, respectively. The terminology includes the above-listed words, derivatives thereof and words of similar import.

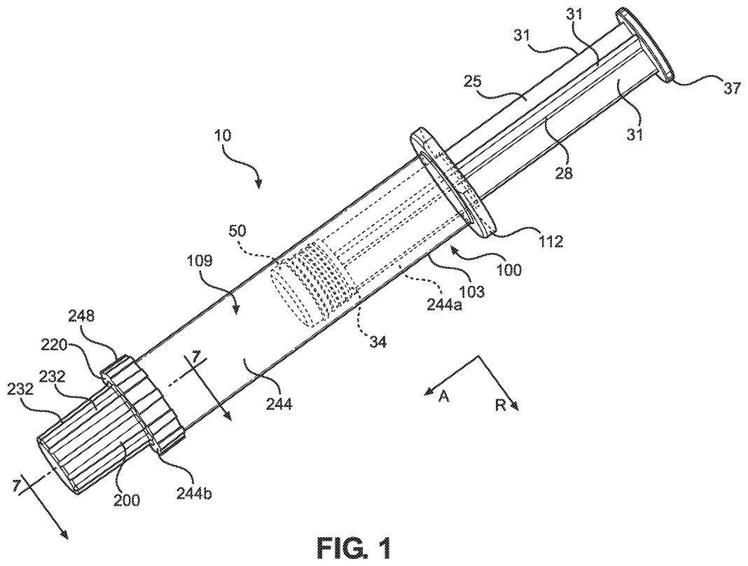

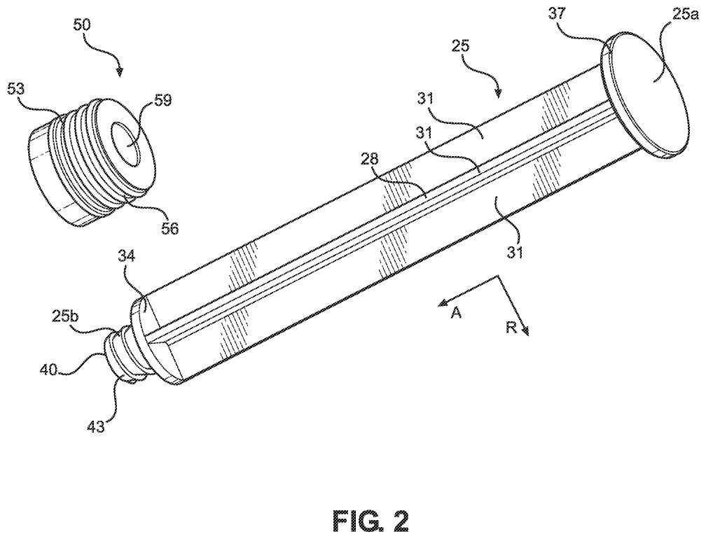

Referring to FIGS. 1-4B, the syringe assembly 10 includes a syringe 100 having a barrel body 103. The barrel body 103 can extend from a proximal end 100a to a distal end 100b along the axial direction A and can be molded from a plastic. In one embodiment, the barrel body 103 can comprise a substantially transparent material, such that a user of the syringe assembly 10 can monitor the material levels within the barrel body 103, though barrel bodies 103 having various levels of opacity are contemplated. The barrel body 103 is depicted as comprising a substantially cylindrical shape, though the present disclosure is not intended to be limited to such. The barrel body 103 can be comprised of cyclic olefin copolymer (COC), cyclic olefin polymer (COP), glass, or various other materials. The barrel body 103 can have an outer surface 103a that extends from the proximal end 100a to the distal end 100b along the axial direction A, an inner surface 103b opposite the outer surface 103a that also extends from the proximal end 100a to the distal end 100b along the axial direction A, and a distal surface 103c that extends along the radial direction R at the distal end 100b of the barrel body 103. The inner surface 103b of the barrel body 103 defines a chamber 109 that extends along the axial direction A from an opening 106 at the proximal end 100a to the Luer connection 120 at the distal end 100b, where the Luer connection 120 will be discussed further below. The chamber 109 is configured to receive and store a material, such as a liquid, for dispensing through the tip 126. The syringe 100 also includes a flange 112 extending radially outwards from the proximal end 100a of the barrel body 103, where the function of the flange 112 will be described further below. Though depicted as defining an oval shape with two flat, oppositely positioned sides, the flange 112 can define other shapes as desired.

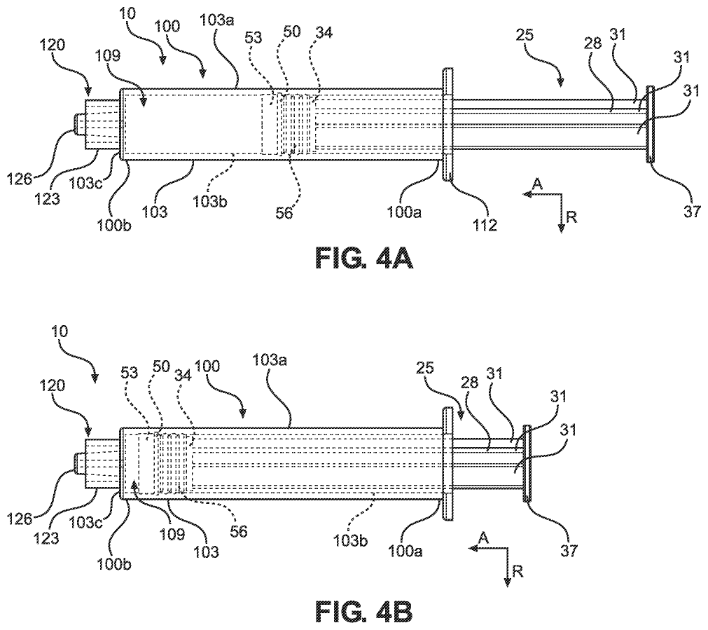

The chamber 109 can be sized and configured to receive a plunger 50, such that the plunger 50 is capable of sliding along the axial direction A through the chamber 109. The plunger is configured to define the proximal-most extent to which the material can travel through the chamber 109. The plunger 50 can have a substantially cylindrical body 53, though the shape of the body 53 will generally conform to the shape of the chamber 109. The body 53 can be comprised of a substantially flexible material such as rubber, though other embodiments are contemplated where the plunger defines other materials. The plunger 50 can further include a plurality of ridges 56 extending radially outwards from the body 53. As shown, the plurality of ridges 56 extend substantially circumferentially around the body 53 and are aligned and spaced apart along the axial direction A. However, the ridges 56 can comprise different sizes, shapes, and arrangements in other embodiments. The ridges 56 can function to engage the inner surface 103b of the barrel body 103 of the syringe 100 so as to create a fluid seal between the plunger 50 and the syringe 100. As the plunger 50 moves distally through the chamber 109 (such as from a first position shown in FIG. 4A to a second position shown in FIG. 4B), the plunger 50 can function to push material out of the chamber 109 through the tip 126. Alternatively, as the plunger 50 moves proximally through the chamber 109, the plunger 50 can function to draw material into the chamber 109 through the tip 126.

The plunger 50 can define a bore 59 that extends into the body 53 from its proximal end. The bore 59 can be configured to engage a portion of a plunger rod 25, which allows a user of the syringe assembly 10 to manually move the plunger 50 through the chamber 109 of the syringe 100 along the axial direction A. The plunger rod 25 extends from a proximal end 25a to a distal end 25b opposite the proximal end 25a along the axial direction A. The plunger rod 25 can comprise a rod body 28 at its center, where the rod body 28 comprises an elongated, axially-extending rod. Connected to the rod body 28, the plunger rod 25 can include a plurality of walls 31 extending radially outwards from the rod body 28. As depicted, each of the walls 31 defines a substantially rectangular body that extends radially outwards from the rod body 28 and axially along the length of the rod body 28. The plunger rod 25 is shown as including four walls 31, where the walls 31 are arranged about the rod body 28 circumferentially spaced apart 90 degrees, such that the arrangement of walls 31 forms a substantially plus-shaped orientation. However, the plunger rod 25 can include more or less walls 31 in other embodiments, and thus other arrangements of walls 31 can define other shapes. Additionally, it is contemplated that the walls 31 can define other shapes or extend to different extents along the axial length of the rod body 28 or radially outwards from the rod body 28. The walls 31 define a height along the radial direction R that is less than the diameter of the chamber 109, so that the plunger rod 25 can freely move along the axial direction A within the chamber 109, and the walls 31 may or may not contact the inner surface 103b of the syringe 100. The walls 31 can function to provide stability and strength to the plunger rod 25, while minimizing the cross-sectional footprint of the plunger rod 25 so as to reduce material requirements for the plunger rod 25, thus reducing overall weight of the syringe assembly 10.

The rod body 28, as well as the walls 31, can extend from a first flange 34 positioned at the distal end 25b of the plunger rod 25 to a second flange 37 positioned at the proximal end 25a of the plunger rod 25. Each of the first and second flanges 34, 37 can be substantially cylindrically shaped, though other shapes are contemplated. The plunger rod 25 can also include a connection extension 40 that extends from the distal end 25b along the axial direction A from the first flange 34 in a direction opposite the rod body 28. The connection extension 40 is configured to be received within the bore 59 of the plunger 50 so as to couple the plunger 50 to the distal end 25b of the plunger rod 25. To strengthen this connection, the connection extension 40 can define a plurality of barbs 43 extending outwards along the radial direction R. The barbs 43 increase in diameter as they extend proximally along the axial length of the connection extension 40. These barbs 43 allow the connection extension 40 to be easily inserted into the bore 59 of the plunger 50 in a first axial direction, but upon attempted removal of the connection extension 40 from the bore 59, the barbs 43 will engage the wall of the bore 59, thus preventing disengagement of the plunger 50 from the plunger rod 25. Though one method of engagement between the plunger 50 and plunger rod 25 is shown, other methods are contemplated, such as forming the plunger 50 onto the plunger rod 25, a simple interference fit, threaded engagement, snap-fit, etc.

Once the plunger 50 and the distal end 25b of the plunger rod 25 are inserted into the chamber 109 of the syringe 100, the proximal end 25a of the plunger rod 25 being located outside the chamber 109, the plunger rod 25 can be used to control dispensing of the material from within the chamber 109. In operation, movement of the plunger rod 25, and thus the plunger 50, distally through the chamber 109 along the axial direction A forces material to flow out of the chamber 109 through the tip 126. To do this, a user can, using one hand, pull the flange 112 of the syringe 100 and the second flange 37 of the plunger rod 25 towards each other. Conversely, movement of the plunger rod 25, and thus the plunger 50, proximally through the chamber 109 along the axial direction A draws material into the chamber 109 through the tip 126. To do this, a user can, using one or two hands, push the flange 112 of the syringe 100 and the second flange 37 of the plunger rod 25 away from each other.

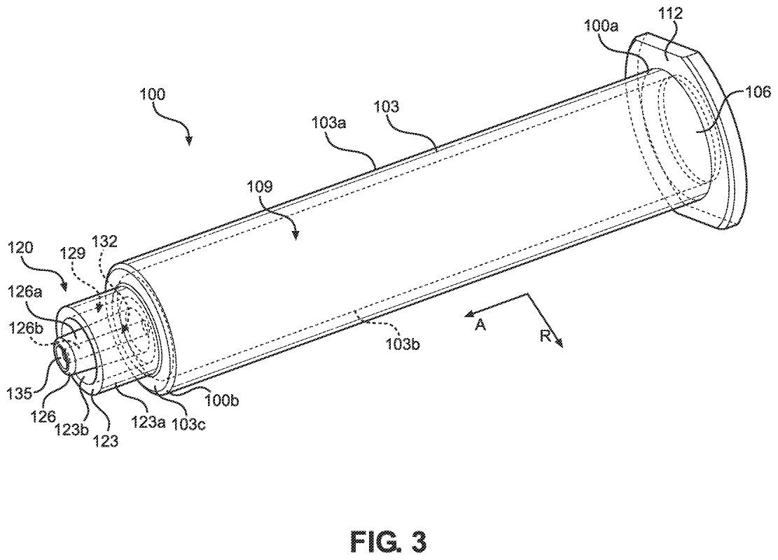

Referring to FIGS. 3-5 and 8, the Luer connection 120 of the syringe 100 will be described in greater detail. The Luer connection 120 can extend from the distal end 100b of the barrel body 103 along the axial direction A. In particular, the Luer connection 120 can extend from the distal surface 103c of the barrel body 103 along the axial direction A. At the center of the Luer connection 120, a tip 126 extends from the distal end 100b of the barrel body 103 along the axial direction A. As depicted, the tip 126 takes the form of a tapered, hollow tube, though other embodiments of the tip 126 are contemplated, such as a cylindrical, hollow tube. The tip 126 has an outer surface 126a and an inner surface 126b opposite the outer surface 126a, where the inner surface 126b defines a passage 132 that extends through the tip 126. The passage 132 can extend from the chamber 109 of the syringe 100 to an outlet 135 of the Luer connection 120. As the passage 132 and the outlet 135 are in fluid communication with the chamber 109, the passage 132 and the outlet 135 thus define the pathway for material being dispensed from the chamber 109 of the syringe 100.

The Luer connection 120 further includes an outer wall 123 that extends from the distal end 100b of the barrel body 103 along the axial direction A. As depicted, the outer wall 123 takes the form of a substantially cylindrical, hollow tube, though other embodiments of the outer wall 123 are contemplated, such as a tapered, hollow tube. The outer wall 123 has an outer surface 123a and an inner surface 123b opposite the outer surface 123a. As depicted, the outer surface 123a is smooth, though other embodiments of the outer surface 123a are contemplated, e.g., ridged or otherwise textured. Similarly, the inner surface 123b is depicted as smooth, though other embodiments of the inner surface 123b also are contemplated, e.g., threaded (female or male). The outer wall 123 extends circumferentially around the tip 126, such that the inner surface 123b of the outer wall 123 faces the outer surface 126a of the tip 126. As a result, a gap 129 can be defined between the outer wall 123 and the tip 126, specifically between the inner surface 123b of the outer wall 123 and the outer surface 126a of the tip 126. The gap 129 can be configured to receive a portion of the tip cap 150, as will be described further below.

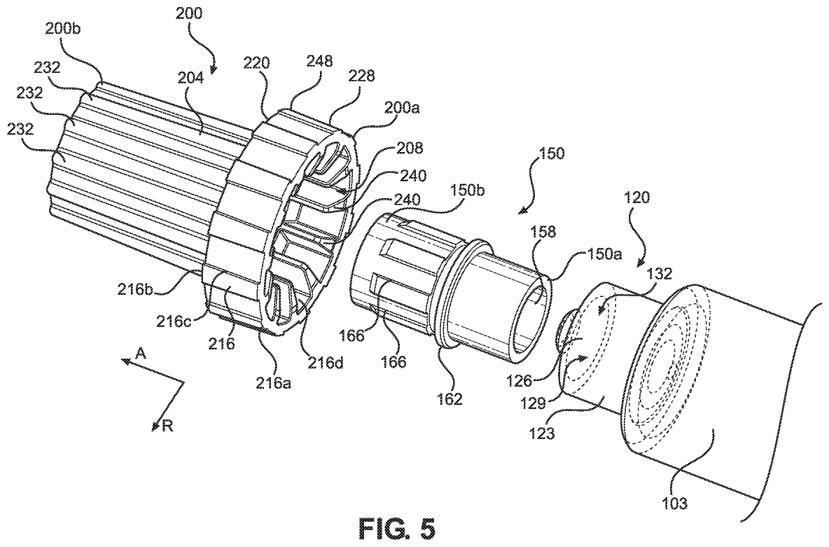

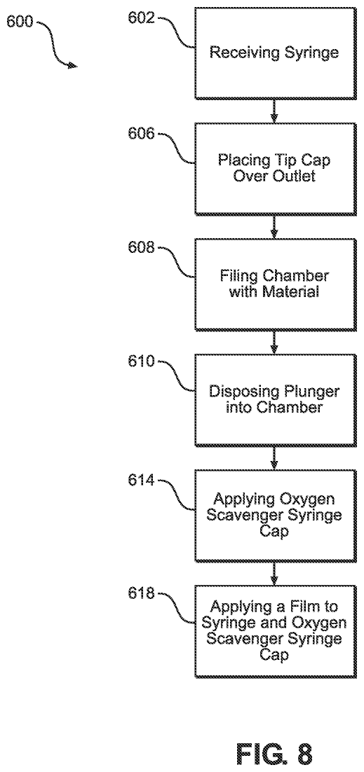

Now referring to FIGS. 5 and 8, the outlet 135 needs to be sealed so as to prevent material from leaking out of the chamber 109. To do this, a tip cap 150 can be attached to the Luer connection 120 so as to seal the outlet 135. The tip cap 150 can extend from a proximal end 150a to a distal end 150b opposite the proximal end 150a along the axial direction A. As depicted, the proximal end 150a defines an opening 158, whereas the distal end 150b is closed. The tip cap 150 can define a central passage 154 extending along the axial direction A into the tip cap 150 from the opening 158. The tip cap 150 can further define a ridge 162 extending radially outwards from the outer surface of the tip cap 150, and substantially continuously around the entirety of the perimeter of the tip cap 150. However, in other embodiments, the ridge 162 may only extend partially around the perimeter of the tip cap 150, or may not be present at all. Further, a plurality of ribs 166 may extend radially outwards from the outer surface of the tip cap 150 distal to the ridge 162. The ribs 166 may be arranged circumferentially around the tip cap 150 so as to provide a texture for grasping by a user of the syringe assembly 10. Though one embodiment of ribs 166 are shown, the present disclosure is not intended to be limited to such.

In operation, as stated previously, the tip cap 150 is configured to be attached to the Luer connection 120 of the syringe 100 so as to create a fluid seal over the outlet 135. To accomplish this, the tip cap 150 can be pushed into the Luer connection 120 with a force along the axial direction A, such that the central passage 154 of the tip cap 150 receives the tip 126 of the Luer connection 120 and the portion of the tip cap 150 proximal to the ridge 162 is disposed in the gap 129 defined between the outer wall 123 and the tip 126. Due to spacing of the outer wall 123 and tip 126 and the thickness of the tip cap 150, as well as the diameter of the passage 132 of the tip cap 150 relative to the diameter of the tip 126, the tip cap 150 can be secured to the Luer connection 120 through an interference fit. In other embodiments, the inner surface 123b comprises threads (female or male), the external portion of the tip cap 150 proximal to the ridge 162 comprises complementary threads, and the tip cap 150 is secured to the Luer connection 120 through a screw-in fit. The ridge 162 can contact the upper surface of the outer wall 123 so as to limit the extent to which the tip 126 can be disposed in the passage 132. Once disposed into the Luer connection 120, the outlet 135 can be located at a distal-most part of the passage 132 and the tip 126 can engage the inner surface of the passage 132, thus creating a fluid seal over the outlet 135.

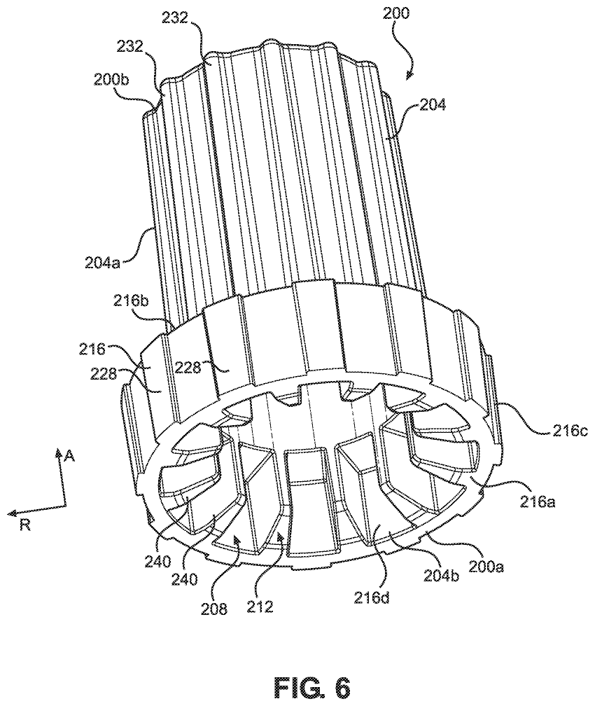

Now referring to FIGS. 1 and 5-7, the oxygen scavenger syringe cap 200 will be discussed in detail. The oxygen scavenger syringe cap 200 can include a main body 204 that extends from a proximal end 200a to a distal end 200b opposite the proximal end 200a along the axial direction A. The main body 204 may be configured as a substantially hollow cylinder, though other shapes are contemplated, as the shape of the oxygen scavenger syringe cap 200 can vary according to the shape of the syringe 100, and specifically the Luer connection 120. The oxygen scavenger syringe cap 200 may be formed through injection molding and may comprise a plastic that is substantially impermeable to oxygen. In some embodiments, the plastic comprises a polymer selected from polypropylene (PP), polycarbonate (PC), high density polyethylene (HDPE), ethylene vinyl alcohol (EVOH), polyamide (PA), polyethylene naphthalate (PEN), polyethylene terephthalate (PET), or a combination thereof. However, other methods of forming the oxygen scavenger syringe cap 200 and other materials for forming the oxygen scavenger syringe cap 200 are contemplated. The main body 204 defines an outer surface 204a and an inner surface 204b opposite the outer surface 204a, where the inner surface 204b defines a passage 208 configured to receive the Luer connection 120 and the tip cap 150. The distal end 200b of the main body 204 can be closed, whereas the proximal end 200a can define an opening 212, where the passage 208 extends from the opening 212 along the axial direction A and terminates within the main body 204 at a location proximal to the distal end 200b. However, in other embodiments the distal end 200b of the main body 204 may be open. In yet other embodiments, the distal end 200b of the main body 204 can be partially closed.

The proximal end 200a of the main body may define a collar 216 that substantially surrounds the Luer connection 120 when the oxygen scavenger syringe cap 200 is disposed over the Luer connection 120, as will be discussed further below. As depicted, the collar 216 defines a substantially annular disc, though it is contemplated that the collar 216 may define other shapes as desired. The collar 216 may have a proximal surface 216a, a distal surface 216b opposite the proximal surface 216a along the axial direction A, an outer surface 216c that extends from the proximal surface 216a to the distal surface 216b, an inner surface 216d opposite the outer surface 216c, and a surface 220 adjacent the distal surface 216b. In the depicted embodiment, the outer surface 216c of the collar 216 can comprise a portion of the outer surface 204a of the main body 204, and the inner surface 216d may comprise a portion of the inner surface 204d of the main body 204. The collar 216 may define a plurality of internal ribs 240 that extend radially inwards from the inner surface 216d and are positioned circumferentially around the inner surface 216d. Though one particular number and arrangement of internal ribs 240 is shown, other numbers and arrangements are contemplated. For example, though the internal ribs 240 are depicted as being substantially equidistantly spaced apart, non-equidistant spacing of the internal ribs 240 is contemplated. In other embodiments, the inner surface 216d is substantially smooth, i.e., lacking any internal ribs.

In operation, the oxygen scavenger syringe cap 200 may be attached to the syringe 100 by pressing the oxygen scavenger syringe cap 200 over the Luer connection 120 and the tip cap 150 via a force applied along the axial direction A. The internal ribs 240 may be configured such that, when this occurs, the internal ribs 240 of the oxygen scavenger syringe cap 200 form an interference fit with the Luer connection 120, specifically the outer surface 123a of the outer wall 123 of the Luer connection 120. This interference fit can cause the oxygen scavenger syringe cap 200, and particularly the proximal end 200a, to resist disengagement from the Luer connection 120 as a result of a distally or rotationally applied force to the oxygen scavenger syringe cap 200. In addition to the interference fit, in other embodiments it is contemplated that the collar 216 can be attached to the Luer connection 120 and/or another portion of the syringe 100 via a sonic weld, adhesive, gripping material, etc. In certain embodiments, the oxygen scavenger syringe cap 200 may be further secured to the Luer connection 120 by a film 244 disposed over the collar 216 or a portion thereof.

In some embodiments, the proximal surface 216a of the collar 216 can abut the distal surface 103c of the syringe 100 when the oxygen scavenger syringe cap 200 is fully disposed over the Luer connection 120. This can function to limit the axial movement of the oxygen scavenger syringe cap 200 in relation to the syringe 100 and indicate to the user of the syringe assembly 10 that the oxygen scavenger syringe cap 200 is fully in place. Notably, the outer diameter of the collar 216 may not extend out past the outer diameter of the outer surface 103a of the barrel body 103, and as well as not extend proximally past the distal surface 103c. As a result, the collar 216 can be spaced in an entirety from the outer surface 103a of the barrel body 103. Because of this, a user of the syringe assembly 10 maintains a complete line of sight to the material within the chamber 109 of the syringe 100, thus allowing the user to be constantly aware of the amount of material within the syringe 100. The collar 216 does not prevent the user from viewing any portion of the material within the chamber 109, as well as the distal end of the plunger 50 to determine whether material is still trapped within the chamber 109 between the plunger 50 and the syringe 100. In other embodiments, the proximal surface 216a of the collar 216 can approach, but not abut, the distal surface 103c of the syringe 100 when the oxygen scavenger syringe cap 200 is fully disposed over the Luer connection 120, thereby forming a gap between the proximal surface 216a of the collar 216 and the distal surface 103c of the syringe 100. In yet other embodiments, the proximal surface 216a of the collar 216 can overlap the distal surface 103c of the syringe 100 when the oxygen scavenger syringe cap 200 is fully disposed over the Luer connection 120.

In certain embodiments, when the oxygen scavenger syringe cap 200 is attached to the Luer connection 120, the oxygen scavenger syringe cap 200 can be spaced in an entirety from the tip cap 150, such that a gap is formed between the oxygen scavenger syringe cap 200 and the tip cap 150 and no portion of the oxygen scavenger syringe cap 200 contacts the tip cap 150. This lack of engagement between the oxygen scavenger syringe cap 200 and the tip cap 150 allows the oxygen scavenger syringe cap 200 to be used simply with existing syringe 100 and tip cap 150 assemblies without interfering with the seal the tip cap 150 creates with the Luer connection 120. Other oxygen scavenger syringe cap designs may require complete redesign of the syringe and/or tip cap, which requires additional tooling for manufacture, thus increasing total manufacturing cost and complexity. The oxygen scavenger syringe cap 200 of the present applications presents none of these difficulties.

However, in other embodiments, a portion of the oxygen scavenger syringe cap 200 may be in contact with the tip cap 150. For example, in some embodiments, the inner surface 204b at the distal end 200b can contact the distal end 150b of the tip cap 150 when the oxygen scavenger syringe cap 200 is fully disposed over the Luer connection 120. Preferably, any contact between the oxygen scavenger syringe cap 200 and the tip cap 150 is such that the fluid seal over the outlet 135 is not compromised by the attachment of the oxygen scavenger syringe cap 200 to the syringe 100 or the decoupling of the oxygen scavenger syringe cap 200 from the Luer connection 120.

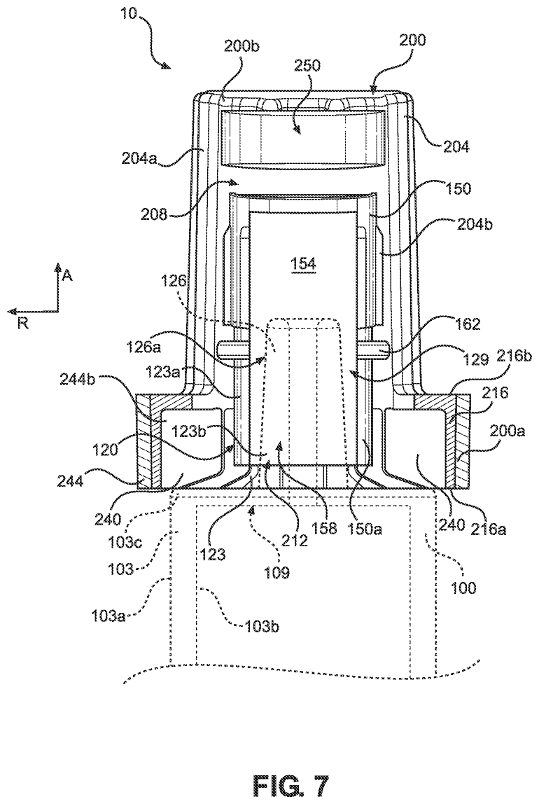

Continuing with FIGS. 1 and 5-7, the distal end 200b of the oxygen scavenger syringe cap 200 includes an oxygen absorber 250. As shown in FIG. 7, the oxygen absorber 250 is a disk, but the oxygen absorber 250 may also be considered as a canister, capsule, sachet, pouch, label, sticker, strip, patch, cartridge, container, or any other reasonable configuration placed in the distal end 200b that allows for the absorption of oxygen from the syringe assembly. In some embodiments, the oxygen absorber 250 may be embedded into the material of the oxygen scavenger syringe cap 200. In other embodiments, the oxygen absorber 250 is attached to at least a portion of the inner surface 204d of the main body 204 by an adhesive. The oxygen absorber 250 may be press fit into the distal end 200b adjacent to the oxygen scavenger syringe cap 200, or may be spaced from the distal end 200b. In some embodiments, the oxygen absorber 250 contains a notch or a recession that is designed to interface with a corresponding recession or notch in the inner surface 204d of the main body 204 to prevent or reduce movement of the oxygen absorber 250 within the main body 204. The oxygen absorber 250 is also spaced from the tip cap 150 such that the oxygen absorber does not come into contact with the distal end 150b of the tip cap 150.

Suitable materials for oxygen absorbers include metal-based substances that remove oxygen by reacting with it by chemical bonding, generally forming a metal oxide component. Metal-based substances include elemental iron as well as iron oxide, iron hydroxide, iron carbide and the like. Other metals for use as oxygen absorbers include nickel, tin, copper, and zinc. Metal-based oxygen absorbers are typically in the form of a powder to increase surface area. Powder formation of the metal-based oxygen absorbers is by any known method including, but not limited to, atomization, milling, pulverization, and electrolysis. Additional materials for oxygen absorbers include low molecular weight organic compounds such as ascorbic acid and analogs thereof, sodium ascorbate, catechol, phenol, activated carbon, and polymeric materials incorporating a resin and a catalyst. In some embodiments of the syringe assembly, the oxygen absorber is a metal-based oxygen absorber. In certain instances of the syringe assembly, the oxygen absorber is an iron-based oxygen absorber. In further instances of the syringe assembly, the oxygen absorber is an iron-based oxygen absorber in the form of a canister. The oxygen absorber 250 may additionally be selected from the group consisting of reduced iron compounds, metal ligands, unsaturated hydrocarbons, and polyamides. In some embodiments, the oxygen absorber 250 is a reduced iron compound. The oxygen absorber 250 may be glued or bonded directly into the distal end 200b of the oxygen scavenger syringe cap 200, or may be embedded within the distal end 200b. Additional configurations may be within the scope of the syringe assembly systems herein.

In some embodiments, the oxygen absorber 250 may reduce the oxygen level in the syringe assembly from the time of packaging to about zero percent in about one to seven days. The capacity of the oxygen absorber 250 to absorb oxygen may depend on the size of the oxygen absorber 250. In some embodiments, the oxygen absorber 250 may have a capacity to absorb about 2 to 10 cc of oxygen/day atm. Another feature of the oxygen absorber 250 is that it may maintain about 0% oxygen level after removal of the initial oxygen in the syringe assembly for an extended period of time. In some embodiments, the oxygen absorber 250 may maintain an about zero oxygen level in the syringe assembly for at least one year, e.g., at least 18 months, at least 24 months, at least 36 months, or at least 48 months. The oxygen absorber 250 may additionally reduce the dissolved oxygen level in the material contained within the syringe assembly 10, especially when the material comprises an oxygen sensitive drug.

The oxygen scavenger syringe cap 200 can also include a plurality of external ribs 228 that extend outwards from the outer surface 204a of the main body 204 at the proximal end 200a along the radial direction. Each of the external ribs 228 can substantially define a rectangular prism, and the external ribs 228 can be equidistantly spaced circumferentially around the proximal end 200a of the main body 204. However, it is contemplated that the external ribs 228 can define alternate shapes, or can be non-equidistantly spaced around the proximal end 200a of the main body 204. The external ribs 228 may be configured to secure a film 244 to the collar 216 of the oxygen scavenger syringe cap 200. The oxygen scavenger syringe cap 200 may also include a plurality of ribs 232 that extend outwards from the outer surface 204a of the main body 204 at the distal end 200b along the radial direction. As depicted, each of the ribs 232 can define a substantially hemispherical extension, and each of the ribs 232 can be equidistantly spaced circumferentially around the distal end 200b of the main body 204. However, it is contemplated that the ribs 232 can define alternate shapes, or can be non-equidistantly spaced around the distal end 200b of the main body 204. The ribs 232 may allow for the distal end 200b of the main body 204 to be easily grasped by a user so as to allow the user to twist the oxygen scavenger syringe cap 200.

Now referring to FIGS. 1 and 7, the syringe assembly 10 can include a film 244 disposed over portions of the syringe assembly 10. The film 244 can define a substantially continuous, solid body and extend from a proximal end 244a to a distal end 244b opposite the proximal end 244a along the axial direction A. In the depicted embodiment, the proximal end 244a of the film 244 can be disposed around a portion of the barrel body 103 of the syringe 100, while the distal end 244b of the film 244 can be disposed around a portion of the oxygen scavenger syringe cap 200. Specifically, the distal end 244b of the film 244 can be disposed around the proximal end 200a of the oxygen scavenger syringe cap 200 to further secure the oxygen scavenger syringe cap 200 to the Luer connection 120. In certain embodiments, the film 244 may extend on the syringe 100 for a length equal or more than 5 mm, e.g., 10 mm, 15 mm, 20 mm, 30 mm, or more, as measured from the distal end 100b of the syringe 100. Alternatively, or additionally, the film 244 may extend onto the syringe 100 for a length equal to or more than 10%, e.g., 20%, 40%, 60%, or more of the length of the barrel body 103. In certain embodiments, the film 244 may extend on the oxygen scavenger syringe cap 200 for a length equal to or more than 2 mm, e.g., 3 mm, 4 mm, 5 mm, 6 mm, or more, as measured from the proximal end 200a. Alternatively, or additionally, the film 244 may extend onto the oxygen scavenger syringe cap 200 for a length equal to or more than 10%, e.g., 20%, 40%, 60%, or more of the length of the oxygen scavenger syringe cap 200. In other embodiments, the film 244 is continuous through at least a distal portion of the syringe 100 and the entire proximal end 200a of the main body 204 of the oxygen scavenger syringe cap 200. In certain embodiments, the film 244 does not extend over the distal end 200b of the main body 204 of the oxygen scavenger syringe cap 200.

The film 244 can be blank, or the film 244 can be printed or written on so as to display information related to the type of the material contained within the chamber 109 of the syringe 100. Examples of the type of information that can be printed on the film 244 includes the material's chemical name, generic name, proprietary name, concentration, total volumetric content within the syringe 100, manufacturer, lot number, date of manufacture, and expiration date. The film 244 can also be bar coded with any combination of this information. The film 244 can be fully transparent, partially transparent, or substantially opaque.

In some embodiments, the film 244 is a heat-shrinkable film made of a thermoplastic material selected from the group consisting of polyvinyl chloride (PVC), ethylene vinyl acetate (EVA), polyethylene terephthalate (PET), oriented polystyrene (OPS), oriented polypropylene (OPP), polylactic acid (PLA) and mixtures thereof. In certain embodiments, the film 244 is made of PVC. In certain embodiments, the interior surface of the film 244 or a portion thereof further comprises an adhesive material, such as a glue or a heat-activated adhesive. In certain embodiments, the interior surface of the distal end 244b of the film 244 can be disposed around a portion of the oxygen scavenger syringe cap 200 comprising an adhesive material. In some embodiments, the film 244 comprises an annular breakable line, such as a dot-line or perforation line, which can be broken in order to facilitate removal of the oxygen scavenger syringe cap 200 from the Luer connection 120.

The film 244 may also include a color-coded portion 248 that is indicative of the type of material contained within the chamber 109 of the syringe 100. This allows the user of the syringe assembly 10 to easily determine what material is within the syringe 100 and helps avoid incorrect medicaments from being mistakenly applied to a patient. In one embodiment, the color-coded portion 248 can comprise a substantially solid band positioned near the distal end 244b of the film 244 and extending circumferentially around the film 244. However, alternative placements, shapes, and sizes of the color-coded portion 248 are contemplated. The color-coded portion 248 can comprise a color selected from a plurality of colors that each correspond to a different material. The relationship between the color of the color-coded portion 248 and the material contained within the syringe 100 can conform to the labeling standards set by ASTM D4774, such that the color-coded portion 248 can be universally recognized and understood within any variety of medical environments. The standards set by ASTM D4774 are shown in the below table. The examples provided for each drug class are exemplary only and not meant to be exhaustive. Drugs that do not fit into the classes shown in Table 1 can be labeled with black printing on a white background under ASTM D4774 standards. Exceptions are noted by the “A” superscript.

TABLE 1
ASTM D4774 Standards

| Drug Class | Examples | Pantone Color |
|---|---|---|
| Induction Agents | Etomidate, Ketamine, Methohexital, Propofol, | Yellow |
| | Thiamylal, Thiopental | |
| Benzodiazepines | Diazepam, Midazolam | Orange 151 |
| Benodiazepine Receptor | Flumazenil | Orange 151 and White Diagonal |
| Antagonist | | Stripes |
| Muscle Relaxants | Succinylcholine^{A} | Fluorescent Red 805 |
| (Depolarizer) | | |
| Muscle Relaxants (Non | Atracurium, Cisatracurium, Mivacurium, | Fluorescent Red 805 |
| Depolarizer) | Pancuronium, Rocuronium, Vecuronium | |
| Relaxant Antagonist | Endophonium, Neostigmine, Pyridostigmine | Fluorescent Red 805 and White |
| (Non-Depolarizer) | | Diagonal Stripes |
| Narcotics | Alfentanil, Fentanyl, Hydromorphone, | Blue 297 |
| | Meperidine, Morphine, Sufentanil, Remifentanil | |
| Narcotic Antagonists | Levallorphan, Naloxone | Blue 297 and White Diagonal |
| | | Stripes |
| Vasopressors | Ephedrine, Norepinephrine, Phenylephrine, | Violet 256 |
| | Epinephrine^{A} | |
| Hypotensive Agents | Hydralazine, Nitroglycerine, Nitroprusside, | Violet 256 and White Diagonal |
| | Phentolamine, Trimethaphan | Stripes |
| Local Anesthetics | Bupivacaine, Chloroprocaine, Lidocaine, | Gray 401 |
| | Mepivacaine, Procaine, Ropivacaine, Tetracaine | |
| Anticholinergic Agents | Atropine, Glycopyrrolate, Scopolamine | Green 367 |
| Beta Blockers | Esmolol, Labetalol, Metroprolol | White Background with Copper |
| | | 876U Bar Across Drug Name |
| Major Tranquilizers and | Droperidol, Inapsine, Haloperidol, | Salmon 156 |
| Anti-Emetics | Levomepromazine, Metoclopramide, | |
| | Ondasetron | |

^{A}Printed against the background color as reversed plate letters with a black bar running from edge to edge of the film

Though the film 244 is described as including a color-coded portion 248, it is also contemplated that in other embodiments all or a portion of the main body 204 of the oxygen scavenger syringe cap 200 defines a color-coded portion that is indicative of the type of material within the chamber 109 of the syringe 100 in combination with or in place of the color-coded portion 248. Like the color-coded portion 248, the color-coded portion of the oxygen scavenger syringe cap 200 can comprise a color selected from a plurality of colors that each correspond to a different material. This can be done through molding the color-coded portion of the oxygen scavenger syringe cap 200 out of a different material or a differently colored variety of the same material as the rest of the oxygen scavenger syringe cap 200. In one embodiment, the collar 216 of the oxygen scavenger syringe cap 200 can define the color-coded portion, though other sections of the oxygen scavenger syringe cap 200 can define the color-coded portion as desired. Like the color-coded portion 248, the color coded portion of the oxygen scavenger syringe cap 200 can conform to the labeling standards set by ASTM D4774.

As stated above, the proximal end 244a of the film 244 can be disposed around a portion of the barrel body 103 of the syringe 100, while the distal end 244b of the film 244 can be disposed around a portion of the oxygen scavenger syringe cap 200. In particular, the external ribs 228 on the proximal end 200a may engage the film 244 so as to ensure that the film 244 retains the proximal end 200a attached to the syringe 100. In one embodiment, this engagement is formed by shrink-wrapping the film 244 over the proximal end 200a of the oxygen scavenger syringe cap 200 and at least a portion of the syringe 100. In other embodiments, the film 244 is adhesive-bonded to the proximal end 200a of the oxygen scavenger syringe cap 200 and at least a portion of the barrel body 103. In yet other embodiments, the film 244 is secured to the proximal end 200a of the oxygen scavenger syringe cap 200 and at least a portion of the barrel body 103 by a combination of shrink-wrapping and adhesive-bonding. In addition to the above-described methods of attaching the film 244 to the oxygen scavenger syringe cap 200 and the syringe 100, various other methods of attaching the film 244 may be utilized as desired.

In certain embodiments, the syringe assembly further comprises a label (not shown) which is attached to at least a portion of the barrel body 103 of the syringe 100. The label can be blank, or the label can be printed or written on so as to display information related to the type of the material contained within the chamber 109 of the syringe 100. Examples of the type of information that can be printed on the label includes the material's chemical name, generic name, proprietary name, concentration, total volumetric content within the syringe 100, manufacturer, lot number, date of manufacture, and expiration date. The label can also be bar coded with any combination of this information. The label can be colored or partially colored, and the label can be fully transparent, partially transparent, or substantially opaque. The label can be comprised of paper, a heat-shrinkable material, an adhesive, or any other suitable materials. In some embodiments, the label is imprisoned between at least a portion of the film 244 and the syringe 100.

Another embodiment, not shown in the figures, of the present disclosure is a pharmaceutical product comprising a syringe assembly 10 and a secondary packaging system therefor. In some embodiments, the secondary packaging is a pouch, blister, flow wrapper, or bag. The secondary packaging can be comprised of an oxygen, light, and/or moisture barrier material, such as high density polyethylene (HDPE), ethylenevinyl alcohol (EVOH), polypropylene (PP), polyethylene terephthalate (PET), polyethylene naphthalate (PEN), and polyamide (PA), metalized film, aluminum foil, oxide coated films, and combinations thereof. In certain embodiments, the secondary packaging system also comprises an oxygen absorber. The oxygen absorber can be a sachet, pouch, canister, capsule, sticker, or strip that is placed inside of the secondary packaging. Alternatively, or additionally, the oxygen absorber can be incorporated into the material of the secondary packaging. In some embodiments, the oxygen absorber is selected from the group consisting of reduced iron compounds, catechol, ascorbic acid and analogs thereof, metal ligands, unsaturated hydrocarbons, and polyamides.

Yet another embodiment of the present disclosure is a pharmaceutical product comprising a plurality of syringe assemblies 10 and a container therefor. In some embodiments, the container is a box, carton, case, package, tray, or tin. Optionally, one or more of the syringe assemblies 10 can be enclosed within a secondary packaging system before being placed into the container. In certain embodiments, each syringe assembly 10 enclosed within the container is filled with the same active ingredient. In other embodiments, each syringe assembly 10 enclosed within the container is filled with a different active ingredient from the same drug class, a different active ingredient from a different drug class, or any combination thereof. For example, the pharmaceutical product can comprise a plurality of syringe assemblies 10 enclosed with a container, wherein the two or more of the syringe assemblies 10 are filled with a different active ingredient from a first drug class, and one or more syringe assemblies 10 are filled with an active ingredient from a second drug class.

Now referring to FIG. 8, a method 600 of filling the syringe 100 with material will be described. At step 602, a syringe 100 can be received, where the syringe 100 has the barrel body 103 extending from a distal end 100b to an open proximal end 100a. In step 602, the syringe 100 can be received with the tip cap 150 placed over the outlet so as to create a fluid seal over the outlet 135. If the syringe 100 is not received with the tip cap 150 placed over the outlet, the tip cap 150 can be placed over the outlet in step 606. At step 608, the method may comprise filling the chamber 109 with the material through the proximal end 100a of the syringe 100. Next, in step 610, the plunger 50 is disposed at the proximal end 100a of the barrel body 103 within the chamber 109. As stated above, the plunger rod 25 is connected to the plunger 50, the plunger rod 25 having a rod body 28 that extends from the proximal end 28a disposed outside the chamber 109 to a distal end 28b disposed within the chamber 109 and connected to the plunger 50. The chamber 109 is defined by the barrel body 103 and extends along the axial direction A, therethrough.

After step 610, step 614 involves applying the oxygen scavenger syringe cap 200 over the Luer connection 120 and the tip cap 150, such that the oxygen scavenger syringe cap 200 is spaced from the tip cap 150. Step 614 may also involve pressing the oxygen scavenger syringe cap 200 over the Luer connection 120 and the tip cap 150 via a force applied along the axial direction A, and such that the oxygen scavenger syringe cap 200 is spaced in an entirety from the tip cap 150. The fluid seal is not compromised when the oxygen scavenger syringe cap 200 is disposed over the Luer connection 120. Step 614 also can include forming an interference fit between the oxygen scavenger syringe cap 200 and the outer surface 123a of the outer wall 123 of the Luer connection. Alternatively, or additionally, step 614 can include engaging a gripping material attached to an inner surface of the oxygen scavenger syringe cap 200 with the Luer connection 120.

In some embodiments, the method 600 may include a step 618, which involves applying a film 244 to the syringe 100 and the oxygen scavenger syringe cap 200 to further maintain the oxygen scavenger syringe cap 200 on the syringe. The film 244 may also prevent axial and/or rotational movement of the oxygen scavenger syringe cap 200 relative to the syringe. Step 618 also may involve bonding a label to the syringe using, for example, heat shrinking, an adhesive, or a combination thereof. The label may be bonded at any suitable location along the syringe. Step 618 may involve any suitable method for bonding the film and label.

The material contained within the chamber 109 of the syringe 100 in the syringe assemblies 10 typically is a liquid, which can be aqueous, non-aqueous, or a combination of aqueous and non-aqueous liquids. In some embodiments, the liquid is a diluent intended for mixing with an active ingredient prior to administration to a subject. Exemplary diluents include, but are not limited to, water, 0.9% saline, 5% dextrose, Ringer's lactate solution, and other pharmaceutically acceptable diluents. In other embodiments, the liquid is a pharmaceutical formulation comprising an active ingredient and, optionally, one or more excipients. Thus, the present disclosure provides a pharmaceutical product comprising a syringe assembly, wherein the liquid is a pharmaceutical formulation. Suitable excipients include, but are not limited to, a tonicity modifier, antioxidant, buffer, pH adjuster, preservative, solubilizer, stabilizer, or a combination of any of the forgoing. A diluent or pharmaceutical formulation can take on any suitable physical form including, but not limited to, solution, suspension, emulsion, or dispersion.

The active ingredient of the pharmaceutical formulation can be a therapeutic agent, a diagnostic agent, a nutrient, or a combination thereof. Examples of therapeutic agents include, but are not limited to antiinfectives, anesthetics, analgesics, anticoagulants, chemotherapeutics, hormones, antihypertensives, antiinflammatories, antiemetics, bronchodilators, adrenergics, immunoglobulins, antipsychotics, antidepressants, and combinations thereof. Examples of diagnostic agents include, but are not limited to x-ray, MRI and ultrasound contrast agents, cholecystokinetics, vasodilators, and combinations thereof. Examples of nutrients include, but are not limited to, salts, carbohydrates, minerals, vitamins, lipids, and combinations thereof.

In some embodiments, the active ingredient is a compound useful for pain management, muscle relaxation, sedation, and/or anesthesia. In certain embodiments, the active ingredient is an opioid, a benzodiazepine, beta blocker, or an α₂-adrenergic receptor agonist. In particular embodiments, the active ingredient is morphine, hydromorphone, hydrocodone, oxycodone, oxymorphone, codeine, buprenorphine, naloxone, naltrexone, fentanyl, remifentanil, sufentanil, alfentanil, meperidine, rocuronium, vecuronium, midazolam, lorazepam, diazepam, neostigmine, atropine, glycopyrrolate, dexmedetomidine, cisastracurium, ropivacaine, lidocaine, propofol, ketamine, succinylcholine, sugammadex, or a combination of the foregoing.

In other embodiments, the active ingredient is promethazine, dopamine, moxifloxacin, linezolid, levofloxacin, danofloxacin, levetiracetam, vancomycin, cefepime, aztreonam, cefoxitin, ceftriaxone, cefazolin, cefotaxime, ceftazidime, gentamicin, oxacillin, nafcillin, erythromycin, penicillin, cyclosporin, cefuroxime, ticarcillin, clavulanic acid, piperacillin, tazobactam, azithromycin, meropenem, ertapenem, tigecycline, micafungin, metronidazole, fluconazole, itraconazole, posaconazole, heparin, enoxaparin, dalteparin, theophylline, acetaminophen (paracetamol), ibuprofen, acetylcysteine, decitabine, azacitidine, docetaxel, pemetrexed, bortezomib, fulvestrant, plerixafor, palonosetron, granisetron, haloperidol, aprepitant, fosaprepitant, dicyclomine, famotidine, amiodarone, nitroglycerin, nicardipine, hydralazine, methyldopate, clevidipine, iloprost, furosemide, dobutamine, esmolol, labetalol, metroprolol, somatropin, lanreotide, liraglutide, abaloparatide, semaglutide, teriparatide, degarelix, sumatriptan, aripiprazole, epinephrine, norepinephrine, ephedrine, pseudoephedrine, phenylephrine, vasopressin, methotrexate, testosterone, estrogen, hydroxyprogesterone, levothyroxine, cetirizine, levocetirizine, diltiazem, verapamil, mexiletine, chlorothiazide, carbamazepine, selegiline, oxybutynin, vitamin A, vitamin B, cyanocobalamin, thiamine, vitamin C, cysteine and tryptophan, or a combination of the foregoing.

While various inventive aspects, concepts and features of the disclosure may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present disclosure. Still further, while various alternative embodiments as to the various aspects, concepts, and features of the disclosure-such as alternative materials, structures, configurations, methods, devices and components, alternatives as to form, fit and function, and so on—may be described herein, such descriptions are not intended to be a complete or exhaustive list of available alternative embodiments, whether presently known or later developed. Additionally, even though some features, concepts or aspects of the disclosure may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features, and concepts may be expressly identified herein as being inventive or forming part of an invention, such identification is not intended to be exclusive, but rather there may be inventive aspects, concepts, and features that are fully described herein without being expressly identified as such or as part of a specific embodiment, the scope of the disclosure instead being set forth in the appended claims or the claims of related or continuing applications. Descriptions of exemplary methods or processes are not limited to inclusion of all steps as being required in all cases, nor is the order that the steps are presented to be construed as required or necessary unless expressly so stated.

While the disclosure is described herein using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in a particular order as desired.

1. A syringe assembly, comprising:
a syringe comprising a barrel body that extends from a proximal end to a distal end and defines a chamber extending along an axial direction therethrough, and a connection extending from the distal end along the axial direction and defining an outlet in fluid communication with the chamber, wherein the chamber contains a material;
a plunger received within the chamber of the syringe to create a fluid seal within the barrel body;
a tip cap defining a central passage configured to receive a portion of the connection such that the tip cap creates a fluid seal over the outlet; and
an oxygen scavenger syringe cap disposed over the connection, the oxygen scavenger syringe cap having a main body that has a proximal end defining an opening, a closed distal end opposite the proximal end along the axial direction, an outer surface, and an inner surface opposite the outer surface that defines a passage configured to receive the tip cap and the connection, wherein the oxygen scavenger syringe cap comprises an oxygen absorber.

2. (canceled)

3. The syringe assembly of claim 1, wherein the oxygen absorber is spaced from the closed distal end of the oxygen scavenger syringe cap and the tip cap.

4. (canceled)

5. The syringe assembly of claim 1, wherein the oxygen absorber is iron-based.

6. The syringe assembly of claim 1, wherein the oxygen scavenger syringe cap comprises an oxygen absorbing material embedded within the closed distal end.

7. The syringe assembly of claim 1, wherein the oxygen scavenger syringe cap comprises a disk or a canister of an oxygen absorbing material adjacent to the closed distal end.

8. (canceled)

9. The syringe assembly of claim 1, wherein the proximal end of the oxygen scavenger syringe cap comprises a flange with an outer diameter larger than an outer diameter of the barrel body of the syringe.

10-13. (canceled)

14. The syringe assembly of claim 1, wherein the fluid seal is not compromised when the oxygen scavenger syringe cap is disposed over the connection.

15. (canceled)

16. The syringe assembly of claim 1, wherein the oxygen scavenger syringe cap is spaced in an entirety from the tip cap.

17. The syringe assembly of claim 1, wherein the material includes an active ingredient that is (a) a therapeutic agent selected from a group consisting of anti-infectives, anesthetics, analgesics, anticoagulants, chemotherapeutics, hormones, antihypertensives, anti-inflammatories, antiemetics, bronchodilators, adrenergics, immunoglobulins, antipsychotics, and antidepressants, or (b) a diagnostic agent selected from a group consisting of x-ray, MRI and ultrasound contrast agents, cholecystokinetics, and vasodilators.

18. The syringe assembly of claim 1, wherein the material includes an active ingredient selected from a group consisting of an opioid, a benzodiazepine, a beta blocker, an α2-adrenergic receptor agonistmorphine, hydromorphone, hydrocodone, oxycodone, oxymorphone, codeine, buprenorphine, naloxone, naltrexone, fentanyl, remifentanil, sufentanil, alfentanil, meperidine, rocuronium, vecuronium, midazolam, lorazepam, diazepam, neostigmine, atropine, glycopyrrolate, dexmedetomidine, cisastracurium, ropivacaine, lidocaine, propofol, ketamine, succinylcholine, sugammadex, promethazine, dopamine, moxifloxacin, linezolid, levofloxacin, danofloxacin, levetiracetam, vancomycin, cefepime, aztreonam, cefoxitin, ceftriaxone, cefazolin, cefotaxime, ceftazidime, gentamicin, oxacillin, nafcillin, erythromycin, penicillin, cyclosporin, cefuroxime, ticarcillin, clavulanic acid, piperacillin, tazobactam, azithromycin, meropenem, ertapenem, tigecycline, micafungin, metronidazole, fluconazole, itraconazole, posaconazole, heparin, enoxaparin, dalteparin, theophylline, acetaminophen (paracetamol), ibuprofen, acetylcysteine, decitabine, azacitidine, docetaxel, pemetrexed, bortezomib, fulvestrant, plerixafor, palonosetron, granisetron, haloperidol, aprepitant, fosaprepitant, dicyclomine, famotidine, amiodarone, nitroglycerin, nicardipine, hydralazine, methyldopate, clevidipine, iloprost, furosemide, dobutamine, esmolol, labetalol, metroprolol, somatropin, lanreotide, liraglutide, abaloparatide, semaglutide, teriparatide, degarelix, sumatriptan, aripiprazole, epinephrine, norepinephrine, ephedrine, pseudoephedrine, phenylephrine, vasopressin, methotrexate, testosterone, estrogen, hydroxyprogesterone, levothyroxine, cetirizine, levocetirizine, diltiazem, verapamil, mexiletine, chlorothiazide, carbamazepine, selegiline, oxybutynin, vitamin A, vitamin B, cyanocobalamin, thiamine, vitamin C, cysteine and tryptophan, or a combination thereof.

19. A method of filling a syringe with a material, the method comprising:
receiving the syringe having a barrel body extending from a distal end having a connection defining an outlet to an open proximal end, the barrel body defining a chamber that extends along an axial direction therethrough, wherein a tip cap is placed over the outlet to create a fluid seal over the outlet;
filling the chamber with the material through the open proximal end;
disposing a plunger within the chamber at the open proximal end; and
applying an oxygen scavenger syringe cap over the connection and the tip cap.

20. The method of claim 19, wherein applying the oxygen scavenger syringe cap includes creating an interference fit between the connection and the oxygen scavenger syringe cap.

21. The method of claim 19, wherein the oxygen scavenger syringe cap comprises an oxygen absorber.

22. The method of claim 19, wherein the oxygen scavenger syringe cap is spaced in an entirety from the tip cap.

23. The method of claim 19, wherein the material includes an active ingredient that is (a) a therapeutic agent selected from a group consisting of anti-infectives, anesthetics, analgesics, anticoagulants, chemotherapeutics, hormones, antihypertensives, anti-inflammatories, antiemetics, bronchodilators, adrenergics, immunoglobulins, antipsychotics, and antidepressants, or (b) a diagnostic agent selected from a group consisting of x-ray, MRI and ultrasound contrast agents, cholecystokinetics, and vasodilators.

24. The method of claim 19, wherein the material includes an active ingredient selected from a group consisting of an opioid, a benzodiazepine, a beta blocker, an α2-adrenergic receptor agonistmorphine, hydromorphone, hydrocodone, oxycodone, oxymorphone, codeine, buprenorphine, naloxone, naltrexone, fentanyl, remifentanil, sufentanil, alfentanil, meperidine, rocuronium, vecuronium, midazolam, lorazepam, diazepam, neostigmine, atropine, glycopyrrolate, dexmedetomidine, cisastracurium, ropivacaine, lidocaine, propofol, ketamine, succinylcholine, sugammadex, promethazine, dopamine, moxifloxacin, linezolid, levofloxacin, danofloxacin, levetiracetam, vancomycin, cefepime, aztreonam, cefoxitin, ceftriaxone, cefazolin, cefotaxime, ceftazidime, gentamicin, oxacillin, nafcillin, erythromycin, penicillin, cyclosporin, cefuroxime, ticarcillin, clavulanic acid, piperacillin, tazobactam, azithromycin, meropenem, ertapenem, tigecycline, micafungin, metronidazole, fluconazole, itraconazole, posaconazole, heparin, enoxaparin, dalteparin, theophylline, acetaminophen (paracetamol), ibuprofen, acetylcysteine, decitabine, azacitidine, docetaxel, pemetrexed, bortezomib, fulvestrant, plerixafor, palonosetron, granisetron, haloperidol, aprepitant, fosaprepitant, dicyclomine, famotidine, amiodarone, nitroglycerin, nicardipine, hydralazine, methyldopate, clevidipine, iloprost, furosemide, dobutamine, esmolol, labetalol, metroprolol, somatropin, lanreotide, liraglutide, abaloparatide, semaglutide, teriparatide, degarelix, sumatriptan, aripiprazole, epinephrine, norepinephrine, ephedrine, pseudoephedrine, phenylephrine, vasopressin, methotrexate, testosterone, estrogen, hydroxyprogesterone, levothyroxine, cetirizine, levocetirizine, diltiazem, verapamil, mexiletine, chlorothiazide, carbamazepine, selegiline, oxybutynin, vitamin A, vitamin B, cyanocobalamin, thiamine, vitamin C, cysteine and tryptophan, or a combination thereof.

25. An oxygen scavenger syringe cap, comprising:
a main body having a proximal end defining an opening, a closed distal end opposite the proximal end along an axial direction, an outer surface, and an inner surface opposite the outer surface, the inner surface defining a passage configured to receive a tip cap such that the tip cap is entirely spaced from the oxygen scavenger syringe cap, and the inner surface is configured to contact a connection of a syringe; and
an oxygen absorber.

26. The oxygen scavenger syringe cap of claim 25, wherein the oxygen absorber is an iron-based oxygen absorber.

27. The oxygen scavenger syringe cap of claim 25, wherein the proximal end of the main body comprises a flange with an outer diameter larger than an outer diameter of a barrel of the syringe.

28. The oxygen scavenger syringe cap of claim 25, wherein the oxygen absorber is adjacent the closed distal end of the main body.
